(19) United States
(12) Patent Application Publication
(10) Pub. No.: US 2018/0049968 A1
(43) Pub. Date: Feb. 22, 2018

(54) INDIVIDUALIZED HAIR TREATMENT

(71) Applicant: The Procter & Gamble Company, Cincinnati, OH (US)

(72) Inventors: Fredy Afif, Frankfurt (DE); Julia Schoell, Stuttgart (DE); Ioannis Constantine Constantinides, Wyoming, OH (US); Kevin Lee Doyle, Fairfield, OH (US); James Daniel Gallagher, Cincinnati, OH (US); Lynn Kristin Stechschulte, Cincinnati, OH (US)

(21) Appl. No.: 15/677,573

(22) Filed: Aug. 15, 2017

Related U.S. Application Data
(60) Provisional application No. 62/376,958, filed on Aug. 19, 2016.

(51) Int. Cl. A61K 8/898; A61K 8/04; A61Q 5/12; A61Q 5/02; A61K 8/25

(52) U.S. Cl. CPC A61K 8/898; A61K 8/04; A61K 8/25; A61Q 5/02; A61Q 5/12

(57) ABSTRACT

The invention relates to a method for individualizing the hair treatment of a consumer by personal supplementation of at least one benefit agent adding further functionality or enhancing existing functionality, or both, of a hair care composition, by the consumer. The method comprises the addition of a hair serum comprising said benefit agent to the hair care composition and the joint application to the hair of the consumer. The invention further relates to the provision of a kit comprising a hair serum and a packaging system.

### FIELD OF THE INVENTION

The present invention relates to a method for individualizing the hair treatment by the consumer. The present invention further relates to a kit for dispensing a hair serum for individualizing the hair treatment for consumers. The present invention also relates to the use of a hair serum in combination with a hair care composition.

### BACKGROUND OF THE INVENTION

It is known in the art that hair care compositions, namely compositions for washing, conditioning or styling the hair, comprise benefit agents that impart specific functionalities. For example, cleanliness, the volume, shine, color, odor, smoothness, moisture, or flexibility of the hair can be addressed by using a respective hair care composition. Other functionalities that can be impacted include the health and sensation of the scalp. In day-to-day hair care, the hair is washed firstly with a shampoo, and subsequently with a conditioner. Both products comprise a benefit agent in a relatively low concentration which helps in keeping the hair looking clean and healthy.

This day-to-day hair care is however limited. The consumer generally uses only a limited number of different shampoos and conditioners at the same time which are selected based on the hair's general needs. Since conventional shampoos and conditioners provide only one specific benefit, the consumer is limited to this one benefit. Further, although the concentration of benefit agents in such compositions should be sufficient to improve the condition of the hair in everyday use, a demand for specifically improved functionalities depending on the daily needs exists.

Thus, consumers have been experimenting with home-made blends of hair treatment or styling products tailored to their hair's needs on a particular day. However, using such blends often results in the hair feeling sticky, and looking limp or dirty. The only fix in such case is to re-wash the hair.

Another approach is the use of third step products like hair masks that need to be worked into the hair after having already used first and second step products, i.e., shampoo and conditioner. However, for working third step products into the hair, additional time and effort is needed. Further, such third step products often result in undesired build-up and heaviness of the hair.

There are methods known in the art of mixing a hair care composition with a hair fiber damage repair benefit agent for treating damaged hair in a repeated process to avoid third step products. However, such methods are limited to the repeated application of the combination of the hair care composition and the hair fiber damage repair benefit agent for treating damaged hair in order to obtain a visible result. Thus, the need for the provision of additional or improved functionalities depending on the daily needs cannot be overcome by such methods.

### SUMMARY OF THE INVENTION

Based on the above, there is still a need to enable consumers to customize their hair care products according to their desired benefit for the day. Further, there is still a need for an easy and quick application of products for boosting specific functionalities in a hair treatment process.

These problems are overcome by the present invention.

According to one aspect of the invention, there is provided a method for individualizing the hair treatment of a consumer by personal supplementation of at least one benefit agent adding further functionality or enhancing existing functionality, or both, of a hair care composition, by the consumer. The method comprises the steps of
(1) defining a desired condition of the consumers hair, determining the existing condition of the consumers hair,
(2) selecting at least one hair serum comprising at least one benefit agent supplementing at least one additional functionality or enhancing at least one existing functionality of a hair care composition, or both, shifting the condition of the hair from the determined condition to the desired condition of the hair of the consumer,
(3) adding the at least one selected hair serum to the hair care composition,
(4) applying the mixture of step (3) to the hair of the consumer, and
(5) optionally, rinsing the hair of the consumer.

The hair care composition is a composition adequate by itself for washing, conditioning or styling the hair comprising at least one benefit agent providing at least one functionality for improving the properties of the hair. The hair serum is a composition comprising at least one benefit agent providing at least one functionality for improving the properties of the hair. The volume of hair serum added in step (3) is smaller than the volume of the hair care composition.

According to another aspect of the invention, there is provided a kit for dispensing a hair serum for individualizing the hair treatment of a consumer. The kit comprises
(a) at least one hair serum, and
(b) at least one packaging system comprising a dosing and dispensing system.

According to another aspect of the invention, there is provided the use of at least one hair serum in combination with a hair care composition for modifying the hair care composition by personal supplementation of at least one benefit agent adding further functionality or enhancing existing functionality, or both, of a hair care composition, by the consumer. The volume of hair serum is smaller than the volume of the hair care composition.

A customization of the benefits is achieved by the consumer-controlled dosing of a hair serum boosting specific functionalities, and the subsequent consumer-controlled ability to mix and match the hair serum with a hair care composition. Benefits provided by the hair serum are for example cleanliness, shine, soft and smooth feel, odor, strength, anti-frizz, damage protection against heat, UV irradiation and colorants, health, sensation, replenishment in internal structures, moisture, and color rejuvenation and retention benefits. Due to a set of different boosters comprising concentrated benefit agents which help to deliver the ultimate level of functionality depending on the hair's unique needs, a customizable system is provided. By choosing the right hair serum, a tailor-made improvement of the condition of the hair depending on the daily needs can be created.

Ease and quickness of the individualization method is achieved by applying both, the hair serum and the hair care composition, simultaneously to the consumer's hair. Thereby, the hair serum is worked into the hair in the same step as the hair care composition. Thus, no need to apply the hair serum in a separate step like third step products exists, resulting in an easy and quick hair treatment process.

### BRIEF DESCRIPTION OF THE DRAWINGS

FIG. 1 is a schematic representation of the method for individualizing the hair treatment of a consumer.

FIG. 2a is an exposed view of a dispenser.

FIG. 2b is a side view of a dispenser.

FIG. 2c is a side view of a dispenser with removed cap.

### DETAILED DESCRIPTION OF THE INVENTION

The present invention relates to a method for individualizing the hair treatment of a consumer by personal supplementation of at least one benefit agent adding further functionality or enhancing existing functionality, or both, of a hair care composition, by the consumer.

### Definitions

“Hair treatment” according to the present invention is the application of at least one hair care composition to the hair.

A “benefit agent” is a compound that provides at least one functionality for improving the properties of the hair. Preferably, a benefit agent is a compound that provides at least one functionality for improving the cleanliness, volume, shine, color, smoothness, moisture, odor, strength, health, protection, or flexibility of the hair and health or sensation of the scalp. Benefit agents according to the present invention include silicones, aminosilicones, fatty alcohols, polymeric resins, polyol carboxylic acid esters, cationic polymers, cationic surfactants, insoluble oils and oil derived materials, sugars, polysaccharides, chelants, benzoic acid derivatives such as hydroxybenzoic acids and derivatives, fatty acid esters, anti-dandruff agents and scalp health agents.

A “hair care composition” according to the present invention is a composition adequate by itself for washing, conditioning or styling the hair that comprises at least one benefit agent. Hair care compositions according to the present invention include shampoos, conditioners (like leave-in and rinse-out conditioners), third step hair care compositions (like hair masks, hair tonics, conditioning sprays, hair oils, hair lotions), styling compositions (like styling creams, waxes, gels, sprays, setting lotions, hair lacquers, hair powders, forming water, styling foams), colorants, and the like.

A “hair serum” according to the present invention is a composition comprising at least one benefit agent. A hair serum according to the present invention is used as a mix-in composition of a hair care composition. It is preferred that the concentration of the at least one benefit agent in the hair serum is higher than the concentration of the at least one benefit agent in the hair care composition.

All percentages are by weight of the total composition, unless specifically stated otherwise. All ratios are weight ratios, unless specifically stated otherwise.

All weights as they pertain to listed ingredients are based on the active level and do not include carriers or by-products that may be included in commercially available materials, unless otherwise specified.

The term “comprising,” as used herein, means that other steps and other ingredients which do not affect the end result may be added.

Unless otherwise indicated, all measurements are understood to be made at about 25° C. and at ambient conditions, wherein “ambient conditions” means conditions under about one atmosphere of pressure and at about 50% relative humidity.

### Method for Individualizing the Hair Treatment

The present invention relates to a method for individualizing the hair treatment of a consumer by personal supplementation of at least one benefit agent adding further functionality or enhancing existing functionality, or both, of a hair care composition, by the consumer.

The method for individualizing the hair treatment of a consumer according to the present invention comprises as a first step (1) the definition of a desired condition of the consumer's hair, and the determination of the existing condition of the consumer's hair.

The existing condition of the consumer's hair can be determined by any analytic method available, for example microscopy, or by its appearance or its feel. Preferably, the existing condition of the consumer's hair is determined by its appearance or its feel. Existing conditions determined by the hair's appearance include shine, color, moisture, frizz, split ends, volume, and the like. Existing conditions determined by the hair's feel include cleanliness, softness, strength, smoothness, stickiness, flexibility, volume, and the like.

Accordingly, the desired condition of the hair relates to at least one condition to be improved with regard to at least one of the determined existing conditions of the consumer's hair.

The method comprises as a second step (2) the selection of at least one hair serum comprising at least one benefit agent supplementing at least one additional functionality or enhancing at least one existing functionality of a hair care composition, or both, shifting the condition of the hair from the determined condition to the desired condition of the hair of the consumer.

Preferably, the benefit agent provides at least one functionality for improving the cleanliness, volume, shine, color, smoothness, moisture, strength, odor, health, protection, or flexibility of the hair and health or sensation of the scalp. The benefit agent can also provide anti-frizz, anti-dandruff, or damage repair properties.

As a third step (3), the method comprises adding the at least one selected hair serum to the hair care composition.

The hair care composition can for example be a shampoo, a conditioner (like leave-in and rinse-out conditioner), a third step hair care composition (like a hair mask, hair tonic, conditioning spray, hair oil, hair lotion), a styling composition (like a styling cream, wax, gel, spray, setting lotion, hair lacquer, hair powder, forming water, styling foam), or a colorant. Preferably, the hair care composition according to the present invention is a shampoo or a conditioner. Most preferred as a hair care composition is a conditioner.

The volume of the hair serum is smaller than the volume of the hair care composition. Preferably, the amount of hair serum is in the range of from 0.1 vol.-% to 90 vol.-%, based on the volume of the hair care product, for example of from 0.2 vol.-% to 50 vol.-% or 0.3 vol.-% to 20 vol.-% or 0.4 to 10 vol.-%. Most preferred are amount of hair serum being in the range of from 0.5 vol.-% to less than 10 vol.-%.

It is preferred that the hair serum is added to the hair care composition immediately before use. “Use” of a hair care composition according to the present invention means working the composition into the hair. “Immediately before use” according to the present invention means no more than 10 min before use. It is however preferred to add the hair serum no more than 5 min before use, for example, no more than 3 min before use or no more than 2 min before use. It is most preferred to add the hair serum less than 1 min before use.

As a fourth step (4), the method comprises applying the composition of step (3) to the hair of the consumer.

The composition of step (3) can be applied by any known methods of working a hair care composition into the hair. For example, the composition of step (3) is applied like a shampoo or a conditioner. Preferably, the composition is disposed on the hair, distributed and massaged in.

As an optional fifth step (5), the method comprises rinsing the hair of the consumer. Rinsing is the final activation step that further distributes the hair serum while preventing the adverse effects of overdosed treatment cocktails by removing all excess of components from the hair.

Whether or not the hair is rinsed depends on the hair care composition to which the hair serum is added. If the hair care composition is a rinse-out composition, e.g., a shampoo or a rinse-out conditioner, the method comprises rinsing the hair of the consumer. If the hair care composition is a leave-in composition, e.g., a leave-in conditioner, the method does not comprise rinsing the hair of the consumer.

The shifting of the condition of the hair from the determined condition before using a hair serum according to the present invention to the desired condition of the hair of a consumer can be determined in accordance with the hair condition determination methods described above, namely by analytic methods, or by the hair's appearance or it feel. Preferably, the condition of the hair after carrying out the method according to the present invention is determined by its appearance or its feel.

### Hair Serum

The method according to the present invention includes the use of at least one hair serum. The hair serum can be in any form. For example, the hair serum can be a solution, an emulsion, a suspension, a dispersion, a foam, a powder, a gel, and the like. Preferably, the hair serum is a solution, a dispersion or an emulsion.

The hair serum according to the present invention comprises at least one benefit agent which provides at least one functionality for improving the characteristics of the hair.

It is preferred than the concentration of the at least one benefit agent in the hair serum is higher than the concentration of the at least one benefit agent in the hair care composition. For example, the concentration of the at least one benefit agent in the hair serum is at least 10 times higher than the concentration of the at least one benefit agent in the hair care composition, or at least 20 times higher, or at least 30 times higher, or at least 50 times higher, or at least 75 times higher. Preferably, the concentration of the at least one benefit agent in the hair serum is at least 100 times higher than the concentration of the at least one benefit agent in the hair care composition.

The benefit agent can be present in a hair serum in an amount of from about 0.5 wt.-% to about 100 wt.-% preferably from about 10 wt.-% to about 85 wt.-% more preferably from about 30 wt.-% to about 70 wt.-% and even more preferably from about 40 wt.-% to about 60 wt.-% by weight of the hair serum.

The functionality provided by the benefit agent of the hair serum can be either an additional functionality with regard to the hair care composition or an existing functionality of the hair care composition.

It is preferred that the amount of hair serum added according to step (3) influences the characteristic of the at least one additional or existing functionality. For example, it is preferred that the characteristic of the at least one additional functionality can be individually metered by the amount of hair serum added according to step (3). In particular, it is preferred that the additional or existing functionality is enhanced with increasing amounts of hair serum.

According to the present invention, the hair serum may comprise one benefit agent. The hair serum may also comprise more than one benefit agent. For example, the hair serum comprises 2, 3, 4, 5 or more than 5 benefit agents.

According to the present invention, each benefit agent provides at least one functionality. For example, the benefit agent may provide one functionality. The benefit agent may also provide 2, 3, 4, 5 or more than 5 functionalities. It is preferred that each benefit agent provides one or two functionalities.

The hair serum may further comprise a carrier. A carrier according to the present invention is liquid. Liquid means being in a liquid state at a pressure of 1013 hPa and a temperature of 20° C. Suitable carriers can dissolve, disperse, or suspense the benefit agents of a hair serum, and form a homogeneous or uniform liquid. In a hair serum comprising a combination of two or more benefit agents, it is also possible that a benefit agent serves as a carrier for the further benefit agents.

Preferably, carriers are selected from the group consisting of silicone carriers, oil carriers and hydrophilic carriers. Examples for silicone carriers are alkyl siloxanes and cycloalkyl siloxanes, preferably cycloalkyl siloxanes, more preferably cyclopentasiloxane. Examples for oil carriers are hydrocarbons, fatty acids, and fatty alcohols. The hydrocarbon chain of the oil carriers preferably has a length of from 8 to 24 carbon atoms, more preferably from 12 to 20 carbon atoms. Examples for hydrophilic carriers are aqueous carriers, monovalent and polyvalent water soluble alcohols, and polyglycols. Aqueous carriers are for example water and water-based buffered solutions, preferably with a pH in the range of from 4 to 9. Preferred monovalent alcohols are ethanol, propanol and isopropanol. Preferred polyvalent alcohols are glycerol, ethylene glycol, propylene glycol. Preferred polyglycols are polyethylene glycol and polypropylene glycol.

The hair serum can also comprise mixtures of two or more carriers, for example combinations of a silicone carrier and an oil carrier, of a silicone carrier and a hydrophilic carrier or of an oil carrier and a hydrophilic carrier. Such carriers can also be combined in one molecule, for example glyceryl monooleate.

Preferably, the carrier is comprised in the hair serum in an amount of at least 10 vol.-%, for example in an amount of from 10 to 90 vol.-% or of from 20 to 80 vol.-% or of from 30 to 70 vol.-%, more preferably in an amount of from 40 to 60 vol.-%, based on the total volume of the hair serum.

According to the present invention, only one hair serum can be used. The method according to the present invention further includes the use of more than one hair serum. For example, the method includes using 2, 3, 4, 5 or more than 5 hair serums, preferably, two hair serums. It is preferred that each hair serum differs in at least one functionality. It is further preferred that each hair serum differs in all functionalities.

According to the present invention, each hair serum is added in individual amounts. Preferably each hair serum is added in an individual amount of from 0.1 vol.-% to 90 vol.-%, based on the volume of the hair care composition, for example of from 0.2 vol.-% to 50 vol.-% or 0.3 vol.-% to 20 vol.-% or 0.4 to 10 vol.-%, most preferably the individual amount of each hair serum is in the range of from 0.5 vol.-% to less than 10 vol.-%. Further, the total volume of hair serums is preferably smaller than the volume of the hair care composition.

Preferably, the hair serum comprises
a. a benefit agent or a combination of two or more benefit agents without a carrier;
b. a solution of a benefit agent or of a combination of two or more benefit agents in a carrier;
c. an emulsion of a benefit agent or of a combination of two or more benefit agents in a carrier; or
d. a dispersion of a benefit agent or of a combination of two or more benefit agents in a carrier, wherein the total amount of benefit agent in the hair serum is at least 10 wt.-%, preferably at least 30 wt.-%, more preferably at least 40 wt.-%.

### Benefit Agent

According to the present invention, the benefit agents comprised in the hair serum and in the hair care composition may be the same or different.

Where the benefit agent comprised in the hair serum is also comprised in the hair care composition, an existing functionality of the hair care composition is enhanced. Where the benefit agent comprised in the hair serum is different from the benefit agent in the hair care composition, an additional functionality is added. However, both effects can be observed, for example, if more than one benefit agent is comprised in the hair serum or due to interactions between the components of the hair serum and the hair care composition with the benefit agent.

Preferably, at least one benefit agent in the hair serum and at least one benefit agent in the hair care composition are the same. For example, one benefit agent comprised in the hair serum and in the hair care composition is the same. It is also in accordance with the invention that 2, 3, 4, 5 or more than 5 benefit agents comprised in the hair serum are also comprised in the hair care composition. Where more than one hair serum is used, it is also in accordance with the present invention that one or more benefit agents of the hair serums are different, but that one or more benefit agents of one or more hair serums are the same as the benefit agent comprised in the hair care composition.

Preferably, the at least one benefit agent in the hair serum and the at least one benefit agent in the hair care composition are different. For example, one benefit agent in the hair serum differs from one benefit agent in the hair care composition. It is however also in accordance with the present invention if more than one benefit agent in the hair serum or, when using more than one hair serum, in the hair serums differs from the at least one benefit agent in the hair care composition.

Benefit agents according to the present invention are silicones, aminosilicones, fatty alcohols, polymeric resins, polyol carboxylic acid esters, cationic polymers, cationic surfactants, insoluble oils and oil derived materials. Preferred benefit agents according to the present invention are silicones, more preferred are low viscosity silicone polymers.

For example, the hair serum according to the present invention comprises at least one low viscosity silicone polymer as a benefit agent, or the hair care composition comprises at least one low viscosity silicone polymer as a benefit agent. Preferably, the hair serum and the hair care composition each comprise at least one low viscosity silicone polymer as a benefit agent.

### Low Viscosity Silicone Polymer

Low viscosity silicone polymers according to the present invention have a viscosity of up to 100,000 mPas. They provide good hair conditioning benefits because of the addition of hydrophobic functionalities, e.g., by quaternary amines, ethylene oxides/propylene oxides. Due to their low viscosity, the low viscosity silicone polymers do not have to be blended with low viscosity diluents and dispersants to allow them to be formulated into products. Low viscosity silicone solvents and diluents can often cause viscosity and stability tradeoffs in shampoo products. However, the viscosity of the low viscosity silicone polymers is low enough for them to be used directly or in emulsion form. The conditioning benefits include for example smooth feel, reduced friction, and prevention of hair damage.

The low viscosity silicone polymers may comprise
(i) at least one quaternary group,
(ii) at least one polyorganosiloxane block,
(iii) at least one polyalkylene oxide structural unit, and
(iv) at least one terminal ester group.

Preferably, the low viscosity silicone polymers may comprise
(i) at least one quaternary amine group,
(ii) at least one polyorganosiloxane block comprising greater than 200 siloxane units, preferably 300 to 600 siloxane units,
(iii) at least one polyalkylene oxide structural unit, preferably an ethylene oxide structural unit or a propylene oxide structural unit, and
(iv) at least one terminal ester group.

According to the present invention, the positive charges can result from the quaternary amine groups. The quaternary ammonium groups can be generated by reacting the di-tertiary amines with an alkylating agents, selected from in particular di-epoxides (sometimes referred to also as bis-epoxides) in the presence of mono carboxylic acids and difunctional dihalogen alkyl compounds.

The counterions can be inorganic anions, such as chloride, bromide, hydrogen sulfate, and sulfate. The counterions can be organic anions, like carboxylates deriving from C₁-C₃₀ carboxylic acids, for example acetate, propionate, octanoatealkylpolyethercarboxylate, alkylsulphonate, arylsulphonate, alkylarylsulphonate, alkylsulphate, alkylpolyethersulphate, phosphates derived from phosphoric acid mono alkyl/aryl ester and phosphoric acid dialkyl/aryl ester, or anions from C₁₀-C₁₈ carboxylic acids, for example decanoate, dodecanoate, tetradecanoate, hexadecanoate, octadecanoate and oleate.

The properties of the low viscosity silicone polymers can be, inter alia, modified based upon the selection of acids used.

Various monofunctional organic acids may be utilized to yield the esters. Exemplary embodiments include C₁-C₃₀ carboxylic acids, for example C₂, C₃, C₈ acids, C₁₀-C₁₈ carboxylic acids, for example C₁₂, C₁₄, C₁₆ acids, saturated, unsaturated and hydroxyl functionalized C₁₈ acids, alkylpolyethercarboxylic acids, alkylsulphonic acids, arylsulphonic acids, alkylarylsulphonic acids, alkylsulphuric acids, alkylpolyethersulphuric acids, phosphoric acid mono alkyl/aryl esters and phosphoric acid dialkyl/aryl esters.

In the low viscosity silicone polymers according to the present invention, the molar ratio of the quaternary ammonium groups (ii) and the terminal ester groups (iv) can be less than 100:10, for example less than 100:15, less than 100:20 or less than 100:30. Preferably, the molar ratio of the quaternary ammonium groups (ii) and the terminal ester groups (iv) is less than 100:50. The ratio can be determined via ¹³C-NMR.

The low viscosity silicone polymers have a viscosity of less than 100,000 mPas (100 Pas) at 20° C. and a shear rate of 0.1 s⁻¹ (plate-plate system, plate diameter 40 mm, gap width 0.5 mm). Preferably, the viscosities of the low viscosity silicone polymers may range from 500 to 100,000 mPas, for example from 500 to 70,000 mPas, or from 500 to 50,000 mPas, or from 500 to 20,000 mPas, more preferably from 500 to 10,000 mPas, and most preferably from 500 to 5,000 mPas determined at 20° C. and a shear rate of 0.1 s^{'1}.

The low viscosity silicone polymers according to the present invention can have the general formulas (Ia) and (Ib):

M-Y—[—(N⁺R₂-T-N⁺R₂)—Y—]ₘ—[—(NR²-A-E-A′-NR²)—Y—]ₖ-M (Ia)

M-Y—[—(N⁺R₂-T-N⁺R₂)—Y—]ₘ—[—(N⁺R²₂-A-E-A′-N⁺R²₂)—Y—]ₖ-M (Ib)

wherein:
m is more than 0, preferably from 0.01 to 100, for example from 0.1 to 100, or from 1 to 100, or from 1 to 50, or from 1 to 20, or from 1 to 10,
k is 0 or from >0 to 50, preferably from 1 to 20, or more preferably from 1 to 10.
   M represents a terminal group, comprising terminal ester groups selected from
—OC(O)—Z
—OS(O)₂—Z
—OS(O₂)O—Z
—OP(O)(O—Z)OH
—OP(O)(O—Z)₂
wherein Z is selected from monovalent organic residues having up to 40 carbon atoms, which may further comprising one or more hetero atoms.

A and A′ each are independently from each other selected from a single bond or a divalent organic group having up to 10 carbon atoms and one or more hetero atoms.

E is a polyalkylene oxide group of the general formula:

—[CH₂CH₂O]_{q}—[CH₂CH(CH₃)O]ᵣ—[CH₂CH(C₂H₅)O]ₛ—

wherein the q, r, and s indices may be defined as follows:
q=0 to 200, preferably from 0 to 100, more preferably from 0 to 50, even more preferably from 0 to 20,
r=0 to 200, preferably from 0 to 100, more preferably from 0 to 50, even more preferably from 0 to 20,
s=0 to 200, preferably from 0 to 100, more preferably from 0 to 50, even more preferably from 0 to 20, and
q+r+s=1 to 600, preferably from 1 to 100, more preferably from 1 to 50, even more preferably from 1 to 40.

R is selected from monovalent organic groups having up to 22 carbon atoms and optionally one or more heteroatoms, wherein the free valencies at the nitrogen atoms are bound to carbon atoms. Preferably, R represents a monovalent straight chain, a cyclic or a branched C₁-C₂₀ hydrocarbon radical, which can be interrupted by one or more —O— or —C(O)— and which can be substituted by —OH.

R² is selected from hydrogen or R.
Y is a group of the formula:

—K—S—K— and -A-E-A′- or -A′-E-A-,

According to the present invention, S is

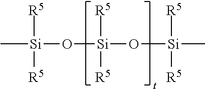

wherein
t is from 200 to 1000, preferably from 300 to 500, and
R⁵ is C₁-C₂₂-alkyl, C₁-C₂₂-fluoralkyl or C₁-C₂₂-fluoraryl. Preferably, R⁵ is C₁-C₁₈-alkyl, C₁-C₁₈-fluoralkyl or C₁-C₁₈-fluoraryl. More preferably, R⁵ is C₁-C₆ alkyl, C₁-C₆ fluoroalkyl, or C₁-C₆ fluoroaryl. Even more preferably R⁵ is C₁-C₄ fluoroalkyl or C₁-C₄ phenyl. Most preferably, R⁵ is methyl, ethyl, trifluoropropyl and phenyl.
The term “C-C₂₂ alkyl” means that the aliphatic hydrocarbon groups possess from 1 to 22 carbon atoms which can be straight chain or branched, for example methyl, ethyl, propyl, n-butyl, pentyl, hexyl, heptyl, nonyl, decyl, undecyl, isopropyl, neopentyl and 1,2,3-trimethyl hexyl.
The term “C₁-C₂₂ fluoroalkyl” means that the aliphatic hydrocarbon compounds possess from 1 to 22 carbon atoms which can be straight chain or branched and which are substituted with at least one fluorine atom, for example monofluormethyl, monofluoroethyl, 1,1,1-trifluorethyl, perfluoroethyl, 1,1,1-trifluoropropyl, 1,2,2-trifluorobutyl.
The term “aryl” means unsubstituted phenyl or phenyl substituted one or more times with OH, F, Cl, CF₃, C₁-C₆ alkyl, C₁-C₆ alkoxy, C₃-C₇ cycloalkyl, C₂-C₆ alkenyl or phenyl. “Aryl” may also mean naphthyl.
If two or more groups S are present in the low viscosity silicone polymer, they can be the same or different.
   K is a bivalent or trivalent straight chain, a cyclic or branched C₂-C₄₀ hydrocarbon residue, preferably a cyclic or branched C₂-C₂₀ hydrocarbon residue, which is optionally interrupted by —O—, —NH—, trivalent N, —NR¹—, —C(O)—, —C(S)—, and optionally substituted with —OH, wherein R¹ is defined as above,
The residues K may be identical or different from each other. In the —K—S—K— moiety, the residue K is bound to the silicon atom of the group S via a C—Si-bond.
A, A′ and E are as defined above.

T is selected from a divalent organic group having up to 20 carbon atoms and one or more hetero atoms. Preferably, T represents a divalent straight-chain, cyclic, or branched C₁-C₂₀ hydrocarbon radical, which can be interrupted by —O— or —C(O)— and which can be substituted by hydroxyl.

Preferably, the repeating units according to formulas (Ia) and (Ib) are in a statistical arrangement, not in a block-wise arrangement.

According to the present invention, the low viscosity silicone polymers may be of the general formulas (IIa) or (IIb):

M-Y—[—N⁺R₂—Y—]ₘ—[—(NR²-A-E-A′-NR²)—Y-]ₖ-M (IIa)

M-Y—[—N⁺R₂—Y—]ₘ—[—(N⁺R²₂-A-E-A′-N⁺R²₂)—Y-]ₖ-M (IIb)
M, Y, R, R², A, A′, E, m, and k are defined as above.
Preferably, the repeating units according to formulas (IIa) and (IIb) are in a statistical arrangement, not in a block-wise arrangement.

In the low viscosity silicone polymers according to formulas (Ia), (Ib), (IIa), and (IIb), the molar ratio of the polyorganosiloxane-containing repeating group —K—S—K— and the polyalkylene repeating group -A-E-A′- or -A′-E-A- can be between 100:1 and 1:100, preferably between 20:1 and 1:20, and more preferably between 10:1 and 1:10.

The low viscosity silicone polymers can be present in a hair serum in an amount of from about 0.5 wt.-% to about 90 wt.-% preferably from about 10 wt.-% to about 80 wt.-% more preferably from about 30 wt.-% to about 70 wt.-% and even more preferably from about 40 wt.-% to about 60 wt.-% by weight of the hair serum.

The low viscosity silicone polymers can be present in a hair care composition in an amount of from about 0.05 wt.-% to about 5 wt.-% preferably from about 0.1 wt.-% to about 4 wt.-% more preferably from about 0.15 wt.-% to about 3 wt.-% and even more preferably from about 0.2 wt.-% to about 2 wt.-% by weight of the hair care composition.

It is most preferred according to the present invention that the hair serum or the hair care composition or both comprise at least one low viscosity silicone polymer comprising
i. one or more quaternary groups, preferably quaternary amine groups,
ii. at least one silicone block comprising greater than 200 siloxane units, preferably 300 to 600 siloxane units,
iii. at least one polyalkylene oxide structural unit, preferably an ethylene oxide structural unit or a propylene oxide structural unit, and
iv. at least one terminal ester group,
   wherein the low viscosity silicone polymer has a viscosity of up to 100,000 mPas, preferably of from 500 to 50,000 mPas, more preferably of from 500 to 5,000 mPas.

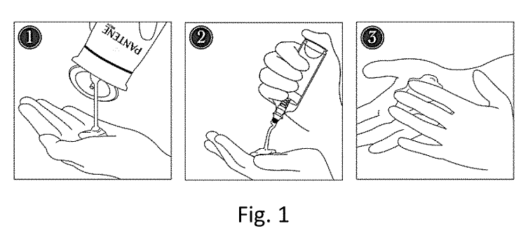

An example of carrying out step (3) of the claimed method is shown in FIG. 1. According to picture 1 of FIG. 1, a desired amount of a hair care composition is applied to the consumer's hand. According to picture 2 of FIG. 1, a desired amount of a selected hair serum is added to the hair care composition in the consumer's hand by using a dispenser. According to picture 3 of FIG. 1, the hair care composition and the hair serum are mixed in the consumer's hands, providing a mixture ready to apply to the consumer's hair (step (4) of the claimed method).

### Kit for Dispensing a Hair Serum for Individualizing the Hair Treatment

A further aspect of the present invention relates to a kit for dispensing a hair serum for individualizing the hair treatment of a consumer comprising
(a) at least one hair serum, and
(b) at least one packaging system comprising a dosing and dispensing system.

The kit may include further components. Examples for further components are hair care compositions, instructions for using the kit, the hair serum or both.

Packaging systems for the hair serum include ampoules, tubes, syringes, bottles, dispensers and the like. Preferred packaging and dosing systems are ampoules, tubes and dispensers. The most preferred packaging systems are dispensers.

With dispensers customized amounts of the hair serum can be dispensed. Preferably the dispensers are airless dispenses, i.e., after using the dispenser no air penetrates the part of dispenser holding the hair serum. Thereby, a longer shelf life of the hair serum after opening can be obtained. Dispensers are preferred where low amounts of hair serum are needed, for example for hair serum with a high benefit agent concentration. Preferably, dispensers include a variable volume dispensing mechanism. The variable volume dispensing mechanism preferably comprises a means for varying the dispensed volumes between 0.01 mL and 10 mL, for example between 0.02 and 5 mL, or between 0.03 and 1 mL, more preferably between 0.05 and 0.5 mL. For dispensing the hair serum from a dispenser, one stroke dispenses the amount of hair serum as set in the variable volume dispensing mechanism. The variable volume dispensing mechanism preferably comprises an indicator indicating to the customer which volume of hair serum is set to be dispensed. Thereby, the customer is free to easily vary the amount of hair serum added to the hair care composition in order to individually meter the additional or existing functionality of the hair care composition. It is therefore easy for the customer to precisely adjust the amount of hair serum dispensed from the dosing system even in these small volume ranges of no more than 10 mL.

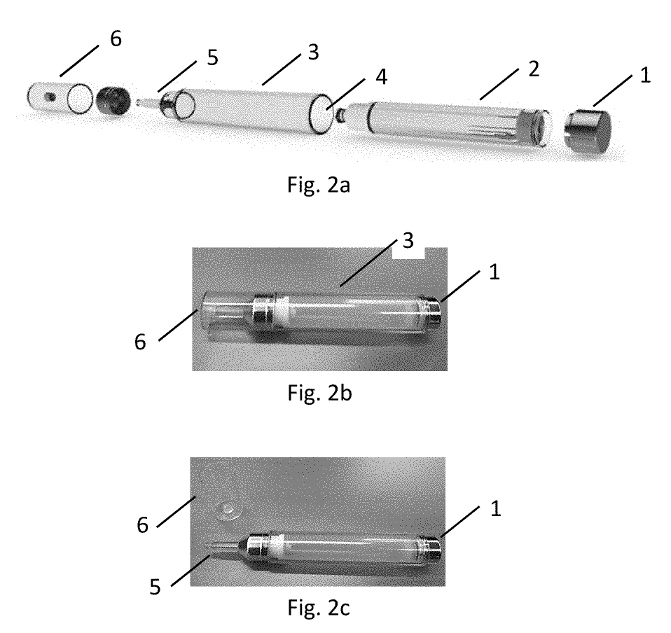

In FIG. 2a-c, a dispenser is shown. The dispenser has a button (1). One stroke of the button (1) initiates the dispensing of an amount of hair serum as set in the variable volume dispensing mechanism. The dispenser comprises a chamber (2) for holding the hair serum. The chamber (2) is airproof. The chamber is disposed in a housing (3). The housing has two openings. Through a first opening (4), the chamber (2) can be inserted into the housing (3). The second opening (5) is the outlet through which the hair serum can be dispensed. Preferably, the second opening (5) is a nozzle. A cap (6) closes the dispenser during storage. The cap (6) can serve as a stand and as means for keeping the second opening (5) clean and protected.

Customized amounts of the hair serum can also be dispensed from tubes. Preferably, tubes and bottles are used where higher amounts of hair serum are needed, for example amounts of from 1 to 30 mL or from 3 to 20 mL, more preferably from 5 to 10 mL. Tubes and bottles are preferably made of a squeezable material. Preferably the squeezable material is a polymeric material, for example comprising polyolefins like polyethylene, high density polyethylene (HDPE), low density polyethylene (LPDE), polypropylene or combinations thereof, polyesters like polyethyleneterephthalate (PET), chlorinated polymers like polyvinylchloride, or combinations thereof.

Ampoules are preferred as packaging systems where each ampoule comprises one serving of the hair serum. The dosing system is therefore an integral part of the ampoule. The consumer does not need to consider any further dosing particulars of the hair serum. Ampoules are preferably made of glass or plastic materials with a predetermined breaking point, preferably at the ampoule neck. For dispensing the hair serum, the ampoule can be opened at the predetermined breaking point, and the hair serum can be poured into the customer's hand. The volume of hair serum in ampoules is preferably in the range of from 0.2 to 15 mL, for example 0.5 to 10 mL, more preferably from 1 to 5 mL.

The hair serum comprised in the kit according to the present invention can be defined as described above with regard to the method for individualizing the hair treatment of a consumer. It is preferred that at least one benefit agent in the hair serum is a low viscosity silicone polymer.

It is particularly preferred that the low viscosity silicone polymer comprises
i. one or more quaternary groups, preferably quaternary amine groups,
ii. at least one silicone block comprising greater than 200 siloxane units, preferably 300 to 600 siloxane units,
iii. at least one polyalkylene oxide structural unit, preferably an ethylene oxide structural unit or a propylene oxide structural unit, and
iv. at least one terminal ester group.

The viscosity of the polymer is up to 100,000 mPas, preferably of from 500 to 50,000 mPas, more preferably of from 500 to 5,000 mPas.

The kit may further comprise
(c) at least one hair care composition,
   wherein a hair care composition is a composition adequate by itself for washing, conditioning or styling the hair comprising at least one benefit agent providing at least one functionality for improving the properties of the hair.

It is preferred that the at least one hair serum (a) and the at least one hair care composition (c) of the kit are stored independently, such that when using the kit, the hair serum and the hair care composition are mixed only when they are dispensed from their packaging.

The hair care composition comprised in the kit according to the present invention can be defined as described above with regard to the method for individualizing the hair treatment of a consumer. It is preferred that at least one benefit agent in the hair care composition is a low viscosity silicone polymer. It is particularly preferred that the low viscosity silicone polymer comprises
i. one or more quaternary groups, preferably quaternary amine groups,
ii. at least one silicone block comprising greater than 200 siloxane units, preferably 300 to 600 siloxane units,
iii. at least one polyalkylene oxide structural unit, preferably an ethylene oxide structural unit or a propylene oxide structural unit, and
iv. at least one terminal ester group.

The viscosity of the polymer is up to 100,000 mPas, preferably of from 500 to 50,000 mPas, more preferably of from 500 to 5,000 mPas.

### Use of the Hair Serum

A further aspect of the present invention relates to the use of at least one hair serum in combination with a hair care composition for modifying the hair care composition by personal supplementation of at least one benefit agent adding further functionality or enhancing existing functionality, or both, of a hair care composition, by the consumer, wherein the volume of hair serum is smaller than the volume of the hair care composition.

Preferably, at least one benefit agent in the hair serum, or at least one benefit agent in the hair care composition, or at least one benefit agent in the hair serum and in the hair care composition is a low viscosity silicone polymer.

It is particularly preferred that the low viscosity silicone polymer comprises
i. one or more quaternary groups, preferably quaternary amine groups,
ii. at least one silicone block comprising greater than 200 siloxane units, preferably 300 to 600 siloxane units,
iii. at least one polyalkylene oxide structural unit, preferably an ethylene oxide structural unit or a propylene oxide structural unit, and
iv. at least one terminal ester group

The viscosity of the polymer is up to 100,000 mPas, preferably of from 500 to 50,000 mPas, more preferably of from 500 to 5,000 mPas.

A further aspect of the present invention relates to the use of a hair serum in combination with a dosing and dispensing system for individualizing the hair treatment for consumers,
wherein a hair serum is a composition comprising at least one benefit agent providing at least one functionality for improving the properties of the hair,
wherein the dosing and dispensing system for the hair serum includes a variable volume dispensing mechanism comprising a means for varying the dispensed volumes between 0.01 mL and 10 mL.

Preferably, the dosing and dispensing system is comprised in a packaging system. More preferably the use of a hair serum in combination with a dosing and dispensing system relates to the use of the kits as described above. Preferably, the dosing and dispensing system is in accordance with the dosing and dispensing system as described with regard to the kits.

The present invention further relates to the use of a kit comprising
(a) at least one hair serum, and
(b) at least one packaging system comprising a dosing and dispensing system,
wherein a hair serum is a composition comprising at least one benefit agent providing at least one functionality for improving the properties of the hair,
wherein the dosing and dispensing system for the hair serum includes a variable volume dispensing mechanism comprising a means for varying the dispensed volumes between 0.01 mL and 10 mL
   in combination with a hair care composition.

Preferably, the kit is characterized by the features as described with regard to the kits.

### EMBODIMENTS OF THE INVENTION

1. Method for individualizing the hair treatment of a consumer by personal supplementation of at least one benefit agent adding further functionality or enhancing existing functionality, or both, of a hair care composition, by the consumer, comprising the steps of
(1) defining a desired condition of the consumers hair, determining the existing condition of the consumers hair,
(2) selecting at least one hair serum comprising at least one benefit agent supplementing at least one additional functionality or enhancing at least one existing functionality of a hair care composition, or both, shifting the condition of the hair from the determined condition to the desired condition of the hair of the consumer,
(3) adding the at least one selected hair serum to the hair care composition,
(4) applying the composition of step (3) to the hair of the consumer, and
(5) optionally, rinsing the hair of the consumer,
wherein a hair care composition is a composition adequate by itself for washing, conditioning or styling the hair comprising at least one benefit agent providing at least one functionality for improving the properties of the hair,
wherein a hair serum is a composition comprising at least one benefit agent providing at least one functionality for improving the properties of the hair, and
wherein the volume of hair serum added in step (3) is smaller than the volume of the hair care composition.

2. Method according to embodiment 1, wherein a benefit agent provides at least one functionality for improving the cleanliness, volume, shine, color, odor, smoothness, moisture, strength, or flexibility of the hair or the health or sensation of the scalp.

3. Method according to embodiment 1 or 2, wherein the hair care composition is a shampoo or a conditioner, preferably a conditioner.

4. Method according to embodiments 1 to 3, wherein the concentration of the at least one benefit agent in the hair serum is at least 10 time higher than the concentration of the at least one benefit agent in the hair care composition, or at least 20 times higher, preferably at least 50 times higher, more preferably at least 100 times higher.

5. Method according to embodiments 1 to 4, wherein the amount of hair serum added in step (3) influences the characteristic of the at least one additional or existing functionality.

6. Method according to embodiment 5, wherein the characteristic of the at least one additional or existing functionality is individually metered by the amount of hair serum added in step (3).

7. Method according to embodiment 5 or 6, wherein the at least one additional functionality is enhanced with increasing amounts of hair serum.

8. Method according to embodiments 5 to 7, wherein the amount of hair serum is in the range of from 0.1 vol.-% to 90 vol.-%, based on the volume of the hair care composition, preferably of from 0.3 vol.-% to 20 vol.-%, more preferably of from 0.5 vol.-% to less than 10 vol.-%.

9. Method according to embodiments 1 to 8, wherein more than one hair serum is used.

10. Method according to embodiment 9, wherein each hair serum differs in at least one functionality.

11. Method according to embodiment 9 or 10, wherein each hair serum is added in individual amounts, preferably each hair serum is added in an individual amount of from 0.1 vol.-% to 90 vol.-%, based on the volume of the hair care composition, preferably of from 0.3 vol.-% to 20 vol.-%, more preferably of from 0.5 vol.-% to less than 10 vol.-%, and wherein the total volume of hair serums is smaller than the volume of the hair care composition.

12. Method according to embodiments 1 to 11, wherein the hair serum is a solution or an emulsion.

13. Method according to embodiments 1 to 12, wherein the at least one benefit agent in the hair serum and the at least one benefit agent in the hair care composition are the same.

14. Method according to embodiments 1 to 13, wherein the at least one benefit agent in the hair serum and the at least one benefit agent in the hair care composition are different.

15. Method according to embodiments 1 to 14, wherein the hair serum comprises at least one low viscosity silicone polymer as a benefit agent.

16. Method according to embodiments 1 to 15, wherein the hair care composition comprises at least one low viscosity silicone polymer as a benefit agent.

17. Method according to embodiments 1 to 6, wherein the hair serum and the hair care composition each comprise at least one low viscosity silicone polymer as a benefit agent.

18. Method according to embodiments 15 to 17, wherein the low viscosity silicone polymer comprises
i. one or more quaternary groups, preferably quaternary amine groups,
ii. at least one silicone block comprising more than 200 siloxane units, preferably 300 to 600 siloxane units,
iii. at least one polyalkylene oxide structural unit, preferably an ethylene oxide structural unit or a propylene oxide structural unit, and
iv. at least one terminal ester group,
wherein the low viscosity silicone polymer has a viscosity of up to 100,000 mPas, preferably of from 500 to 50,000 mPas, more preferably of from 500 to 5,000 mPas.

19. Method according to embodiments 1 to 8, wherein in step (1) the consumer determines the condition of the hair by its feel or its appearance.

20. Kit for dispensing a hair serum for individualizing the hair treatment of a consumer comprising
(a) at least one hair serum, and
(b) at least one packaging system comprising a dosing and dispensing system,
wherein a hair serum is a composition comprising at least one benefit agent providing at least one functionality for improving the properties of the hair,
wherein the dosing and dispensing system for the hair serum includes a variable volume dispensing mechanism comprising a means for varying the dispensed volumes between 0.01 mL and 10 mL.

21. The kit according to embodiment 20, wherein the packaging system is a dispenser.

22. The kit according to embodiment 20, wherein at least one benefit agent in the hair serum is a low viscosity silicone polymer.

23. The kit according to embodiment 21, wherein the low viscosity silicone polymer comprises
i. one or more quaternary groups, preferably quaternary amine groups,
ii. at least one silicone block comprising more than 200 siloxane units, preferably 300 to 600 siloxane units,
iii. at least one polyalkylene oxide structural unit, preferably an ethylene oxide structural unit or a propylene oxide structural unit, and
iv. at least one terminal ester group,
wherein the low viscosity silicone polymer has a viscosity of up to 100,000 mPas, preferably of from 500 to 50,000 mPas, more preferably of from 500 to 5,000 mPas.

24. The kit according to any of embodiments 20 to 23 further comprising
(c) at least one hair care composition,
wherein a hair care composition is a composition adequate by itself for washing, conditioning or styling the hair comprising at least one benefit agent providing at least one functionality for improving the properties of the hair.

25. Use of at least one hair serum in combination with a hair care composition for modifying the hair care composition by personal supplementation of at least one benefit agent adding further functionality or enhancing existing functionality, or both, of a hair care composition, by the consumer,
wherein a hair care composition is a composition adequate by itself for washing, conditioning or styling the hair comprising at least one benefit agent providing at least one functionality for improving the properties of the hair,
wherein a hair serum is a composition comprising at least one benefit agent providing at least one functionality for improving the properties of the hair, and
wherein the volume of hair serum is smaller than the volume of the hair care composition.

26. Use according to embodiment 25, wherein at least one benefit agent in the hair serum, or at least one benefit agent in the hair care composition, or at least one benefit agent in the hair serum and in the hair care composition is a low viscosity silicone polymer.

27. Use according to embodiment 26, wherein the low viscosity silicone polymer comprises
i. one or more quaternary groups, preferably quaternary amine groups,
ii. at least one silicone block comprising more than 200 siloxane units, preferably 300 to 600 siloxane units,
iii. at least one polyalkylene oxide structural unit, preferably an ethylene oxide structural unit or a propylene oxide structural unit, and
iv. at least one terminal ester group,
   wherein the low viscosity silicone polymer has a viscosity of up to 100,000 mPas, preferably of from 500 to 50,000 mPas, more preferably of from 500 to 5,000 mPas.

28. Use of a hair serum in combination with a dosing and dispensing system for individualizing the hair treatment for consumers,
wherein a hair serum is a composition comprising at least one benefit agent providing at least one functionality for improving the properties of the hair,
wherein the dosing and dispensing system for the hair serum includes a variable volume dispensing mechanism comprising a means for varying the dispensed volumes between 0.01 mL and 10 mL.

### EXAMPLES

The compositions illustrated in the following examples are prepared by conventional formulation and mixing methods. All exemplified amounts are listed as weight percent and exclude minor materials such as diluents, preservatives, color solutions, imagery ingredients, botanicals, and so forth, unless otherwise specified.

### I. Individualized Hair Treatment

A customary conditioner is dispensed into the hand of a customer. A hair serum is added as an emulsion directly to the conditioner. The amounts of conditioner and hair serum are given in table 1.

TABLE 1

| Conditioner [mL] | Hair Serum [mL] | |
|---|---|---|
| 15 | 0.5 | 3.3% |
| 15 | 1.0 | 6.7% |
| 15 | 1.2 | 8.0% |
| 15 | 1.5 | 10% |

The combined product is distributed evenly in the hair and left therein for 3 min Subsequently, the hair is thoroughly rinsed with water for 30 seconds. Rinsing is the final activation step that distributes the hair serum while preventing the adverse effects of overdosed treatment cocktails.

For all mixtures of conditioner and hair serum according to table 1, the following results are achieved:
1. The hair serum is easy to mix with the conditioner.
2. The hair serum can be easily applied to and worked into the hair.
3. The wet hair feels clean and conditioned (smooth and tangle free) without residues and build-up on the hair.

### II. Shampoo and Conditioner Treatment Method

Moderately oxidized hair swatches are used for the evaluation. Each hair swatch weighs 2 g and its length is 15.24 cm. Each swatch is treated according to the following protocol:
1. An amount of 0.1 g of shampoo of Example A per gram of hair is applied on a wet hair swatch. Then, the shampoo is milked for 30 seconds followed by rinsing for 30 seconds with water. The excess water is squeezed from the washed hair swatches. The process is repeated so that each swatch is washed twice with the shampoo of Example A.
2. An amount of 0.19 g of the conditioner composition of Example B is mixed with 0.01 g of a hair serum. The combination of conditioner and hair serum is applied on a hair swatch, milked for 30 seconds, rinsed with water for 30 seconds, and combed.
3. A sensory expert evaluates the ease of wet combing according to the scale provided below.
4. The hair swatch is dried in a heat box at 60° C. until dry.
5. A sensory expert evaluates the ease of dry combing according to the scale.

The final rating is the average of the evaluation for three separately treated swatches.

As a control treatment, the protocol is repeated following the same protocol as described above, but the amount of conditioner is 0.2 g for each swatch. No hair serum is added.

### Hair Serum 1: Silicone Booster

TABLE 2

| | wt.-% |
|---|---|
| Dimethiconol solution in cyclopentasiloxane¹ | 90.0 |
| Isosdodecane | 9.2 |
| Tocopherol acetate | 0.2 |
| Tethylhexyl methoxycinnamate | 0.1 |
| Fragrance | 0.5 |

¹Supplied by Dow Corning as DC1501 as a 13.5% solution

### Hair Serum 2: Lipid Booster

TABLE 3

| | wt.-% |
|---|---|
| Oleic acid (mixture with other unsaturated | 76.3 |
| C16-18 and C18 acids)¹ | |
| Glyceryl monooleate² | 23.7 |

¹Supplied by Cremer as OLS-70 Oleic
²Supplied by Cognis as Monomuls 90-O

### Composition A: Shampoo Composition

TABLE 4

| | wt.-% |
|---|---|
| Sodium Laureth-3 Sulfate | 7 |
| Sodium Lauryl Sulfate | 7 |
| Cocoamidopropyl Betaine | 2 |
| Cocamide MEA | 0.5 |
| Citric Acid | 1.11 |
| Sodium chloride | 0.7 |
| Perfume | 0.46 |
| Tetrasodium EDTA | 0.14 |
| DMDM Hydantoin | 0.1 |
| Kathon CG¹ | 0.03 |
| Distilled water | Q.S. to 100% |

¹active components: methylchloroisothiazolinone and methylisothiazolinone

### Composition B: Conditioner Composition

TABLE 5

| | wt.-% |
|---|---|
| Stearyl alcohol | 4.18 |
| Cetyl alcohol | 1.67 |
| Behenyltrimonium chloride | 2.25 |
| Isopropanol | 0.60 |
| Silicone Quaternium-26¹ | 2.65 |
| Benzyl alcohol | 0.40 |
| Disodium EDTA | 0.13 |
| Kathon CG² | 0.03 |
| Perfume | 0.60 |
| Citric acid | Adjust pH to 5 |
| Deionized water | Q.S. to 100% |

¹Supplied by Momentive Performance Materials Inc.
²active components: methylchloroisothiazolinone and methylisothiazolinone)

### Treatment Evaluation Results

TABLE 6

| | Wet | Dry |
|---|---|---|
| | Combing | Combing |
| | Evaluation | Evaluation |
| PROTOCOL | Rating | Rating |
| Composition A/Composition B | 3 | 3 |
| Composition A/Composition B + Hair Serum 1 | 4 | 5 |
| Composition A/Composition B + Hair Serum 2 | 4 | 5 |

1: Poor;
2: Fair;
3: Good;
4: Very good;
5: Excellent

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. Method for individualizing the hair treatment of a consumer by personal supplementation of at least one benefit agent adding further functionality or enhancing existing functionality, or both, of a hair care composition, by the consumer, comprising the steps of
(1) defining a desired condition of the consumers hair, determining the existing condition of the consumers hair,
(2) selecting at least one hair serum comprising at least one benefit agent supplementing at least one additional functionality or enhancing at least one existing functionality of a hair care composition, or both, shifting the condition of the hair from the determined condition to the desired condition of the hair of the consumer,
(3) adding the at least one selected hair serum to the hair care composition,
(4) applying the composition of step (3) to the hair of the consumer, and
(5) rinsing the hair of the consumer,
wherein a hair care composition is a composition adequate by itself for washing, conditioning or styling the hair comprising at least one benefit agent providing at least one functionality for improving the properties of the hair,
wherein a hair serum is a composition comprising at least one benefit agent providing at least one functionality for improving the properties of the hair, and
wherein the volume of hair serum added in step (3) is smaller than the volume of the hair care composition.

2. Method according to claim 1, wherein the hair care composition is a shampoo or a conditioner.

3. Method according to claim 1, wherein the concentration of the at least one benefit agent in the hair serum is at least about 10 times higher than the concentration of the at least one benefit agent in the hair care composition, or at least about 20 times higher, or at least about 50 times higher, or at least about 100 times higher.

4. Method according to claim 1, wherein the amount of hair serum added in step (3) influences the characteristic of the at least one additional or existing functionality.

5. Method according to claim 4, wherein the amount of hair serum is in the range of from about 0.1 vol.-% to about 90 vol.-%, based on the volume of the hair care composition.

6. Method according to claim 1, wherein the hair serum is a solution or an emulsion.

7. Method according to claim 1, wherein the hair serum comprises at least one low viscosity silicone polymer as a benefit agent.

8. Method according to claim 1, wherein the hair care composition comprises at least one low viscosity silicone polymer as a benefit agent.

9. Method according to claim 1, wherein the hair serum and the hair care composition each comprise at least one low viscosity silicone polymer as a benefit agent.

10. Method according to claim 7, wherein the low viscosity silicone polymer comprises
i. one or more quaternary groups,
ii. at least one silicone block comprising more than about 200 siloxane units,
iii. at least one polyalkylene oxide structural unit, and
iv. at least one terminal ester group,
wherein the low viscosity silicone polymer has a viscosity of up to about 100,000 mPas.

11. A kit for dispensing a hair serum for individualizing the hair treatment for consumers comprising
(a) at least one hair serum, and
(b) at least one packaging system comprising a dosing and dispensing system,
wherein a hair serum is a composition comprising at least one benefit agent providing at least one functionality for improving the properties of the hair,
wherein the dosing and dispensing system for the hair serum includes a variable volume dispensing mechanism comprising a means for varying the dispensed volumes between about 0.01 mL and about 10 mL.

12. The kit according to claim 11, wherein the packaging system is a dispenser.

13. The kit according to claim 11, wherein at least one benefit agent in the hair serum is a low viscosity silicone polymer.

14. The kit according to claim 13, wherein the low viscosity silicone polymer comprises
i. one or more quaternary groups, preferably quaternary amine groups,
ii. at least one silicone block comprising more than 200 siloxane units, preferably 300 to 600 siloxane units,
iii. at least one polyalkylene oxide structural unit, preferably an ethylene oxide structural unit or a propylene oxide structural unit, and
iv. at least one terminal ester group,
wherein the low viscosity silicone polymer has a viscosity of up to 100,000 mPas, preferably of from 500 to 50,000 mPas, more preferably of from 500 to 5,000 mPas.

15. The kit according to any of claim 11 further comprising
(c) at least one hair care composition,
wherein a hair care composition is a composition adequate by itself for washing, conditioning or styling the hair comprising at least one benefit agent providing at least one functionality for improving the properties of the hair.
